(19) United States
(12) Patent Application Publication
(10) Pub. No.: US 2026/0174718 A1
(43) Pub. Date: Jun. 25, 2026

(54) MODULATORS OF mTORC1 ACTIVITY AND USES THEREOF

(71) Applicant: Navitor Pharmaceuticals, Inc., Boston, MA (US)

(72) Inventor: Eddine Saiah, Brookline, MA (US)

(21) Appl. No.: 19/125,977

(22) PCT Filed: Nov. 1, 2023
(86) PCT No.: PCT/US2023/036588

§ 371 Date: Apr. 30, 2025

Related U.S. Application Data
(60) Provisional application No. 63/421,288, filed on Nov. 1, 2022.

(51) Int. Cl. A61K 31/198; A61K 9/00; A61K 9/14; A61K 33/14

(52) U.S. Cl. CPC A61K 31/198; A61K 9/0053; A61K 9/141; A61K 33/14

(57) ABSTRACT

The present invention relates to compounds and methods useful for selectively modulating mTORC1 activity.

### CROSS REFERENCE TO RELATED APPLICATIONS

This application claims the benefit of U.S. Provisional App. No. 63/421,288, filed on Nov. 1, 2022, the content of which is hereby incorporated by reference.

### BACKGROUND OF THE INVENTION

Depression in individuals with either major depressive disorder (MDD) and bipolar disorder (BD) are leading causes disease burden. In published PCT application WO 2017/070518, it was found that small molecule mTORC1 activators have a benefit in depressive disorders and other CNS related diseases. Activation of mTORC1 and subsequent synaptogenesis in the prefrontal cortex (PFC) have been suggested to mediate rapid antidepressant effects of pour small molecule mTORC1 activators. Through activation of the mTOR signaling pathway, administration of an mTORC1 activator increased levels of synaptic proteins and dendritic spine density.

Separately, it is known that the mood stabilizer lithium has antisuicidal properties and holds promise for treating other neurological and neurodegenerative diseases.

It has also been shown by Chiu et al. (International Journal of Neuropsychopharmacology, 2015, 1-13) that subtherapeutic (600 mg/L) lithium-pretreated mice exhibited an antidepressant-like response to an ineffective ketamine (2.5 mg/kg, intraperitoneally) challenge in the forced swim test. Both the antidepressant-like effects and restoration of dendritic spine density in the medial prefrontal cortex of stressed mice induced by a single ketamine (50 mg/kg) injection were sustained by post-ketamine treatment with 1200 mg/L of lithium for at least 2 weeks. These benefits of lithium treatments were associated with activation of the mammalian target of rapamycin/brain-derived neurotrophic factor signaling pathways in the prefrontal cortex.

There is urgent and compelling unmet medical need for more effective treatments for diseases, disorders or conditions associated with mTORC1

### DETAILED DESCRIPTION OF CERTAIN EMBODIMENTS

### 1. General Description of Certain Embodiments of the Invention

Among the various aspects of the present invention may be noted the provision of compositions containing lithium salts and organic molecules in a stoichiometric ratio.

In the present invention, a compound comprising a lithium salt and mTORC1 activator in a stoichiometric ratio or co-crystal form are described. Such mTORC1 activators could be an amino acid as described in published PCT application WO 2017/070518. In some embodiments, a compound comprising a lithium salt and an mTORC1 activator has improved efficacy due to the synergetic effect of lithium and the mTORC1 activator. In some embodiments, such derivatives have efficacy at lower therapeutic dose than either single agent administered alone.

In some embodiments, a compound comprising a lithium salt and mTORC1 activator is a co-crystal comprising a lithium salt and mTORC1 activator in a stoichiometric ratio form are described. Such mTORC1 activators could be an amino acid as described in published PCT application WO 2017/070518. In some embodiments, a lithium salt and an mTORC1 activator as a co-crystal has improved efficacy due to the synergetic effect of lithium and the mTORC1 activator. In some embodiments, such derivatives have efficacy at lower therapeutic dose than either single agent administered alone.

U.S. Pat. No. 10,100,066 (“the '066 patent”) filed Oct. 21, 2016 as U.S. Pat. App. Serial No. U.S. Ser. No. 15/331,362 and published as U.S. Pat. App. Pub. No. U.S. 2017/0114080 (“the '080 publication”), the entirety of each is incorporated herein by reference), describe certain mTORC1 modulating compounds. Such compounds include compound I:

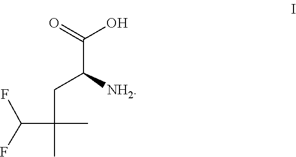

Compound I, (S)-2-amino-5,5-difluoro-4,4-dimethylpentanoic acid, is described in both the '066 patent and the '080 publication. The synthesis of compound I is described in detail at Example 90 of the '066 patent and '080 publication.

It has now been found that a compound comprising lithium chloride and compound I, such as a salt or co-crystal comprising lithium chloride and compound I, and compositions thereof, are useful for treating, preventing, and/or reducing a risk of a disease, disorder, or condition mediated by mTORC1.

One aspect of the present invention is a compound having the formula LiCl*compound I, or a solvate or hydrate thereof, wherein compound I has the structure:

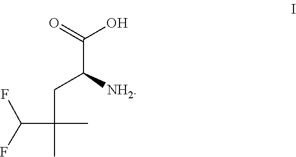

One aspect of the present invention is further directed to a pharmaceutical composition comprising a compound having the formula LiCl*compound I, or a solvate or hydrate thereof. The present invention is further directed to a dosage unit form, the dosage unit form comprising a compound having the formula LiCl*compound I, or a solvate or hydrate thereof. The compound having the formula LiCl*compound I may be a salt or co-crystal that may also include one or more solvate or water molecules in the crystalline lattice.

The present invention is further directed to a method for preparing salts or co-crystals comprising a lithium salt and an organic compound in a stoichiometric ratio. The method comprises dissolving the lithium salt and the organic compound in a solvent and evaporating or cooling the solvent. In one embodiment, the stoichiometric ratio of organic compound to lithium salt is 1:1, respectively.

Other aspects and objects of the invention will be in part apparent and in part pointed out hereinafter.

In some embodiments, provided is a compound having the formula LiCl*compound I. or a solvate or hydrate thereof, wherein compound I has the structure:

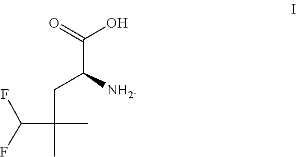

In some embodiments, a compound having the formula LiCl*compound I is a co-crystal.

In some embodiments, the lithium and the compound I are in a stoichiometric ratio of about 1 to about 1.

In some embodiments, provided is a pharmaceutical composition comprising the a compound having the formula LiCl*compound I and a pharmaceutically acceptable carrier, adjuvant, or vehicle.

In some embodiments, the pharmaceutical composition is formulated for oral administration.

In some embodiments, the pharmaceutical composition further comprises an additional therapeutic agent.

In some embodiments, provided is a method of treating, preventing, and/or reducing a risk of a disease, disorder, or condition mediated by mTORC1 in a patient comprising administering to a patient in need thereof an effective amount of a compound having the formula LiCl*compound I or a pharmaceutical composition comprising a compound having the formula LiCl*compound I.

In some embodiments, the disease, disorder, or condition mediated by mTORC1 is depression, bipolar disorder, schizophrenia, chronic unpredictable stress, autism, lysosomal storage disease, Batten disease, cystinosis, Fabry disease, mucolipidosis, mental retardation, anorexia, bulimia, anemia, neutropenia, headache, alcoholism, post-traumatic stress disorder (PTSD), epilepsy, diabetes, liver disease, kidney disorders, arthritis, skin conditions such as seborrhea, overactive thyroid, asthma, Huntington's disease, Graves' disease, herpes simplex, movement disorders such as tardive dyskinesia, Tourette's syndrome, cyclical vomiting, Meniere's disease, tingling or crawling sensation in the skin (paresthesias), or aggressive behavior in attention deficit-hyperactivity disorder (ADHD).

In some embodiments, provided is a method of treating treatment-resistant depression in a patient in need thereof, said method comprising administering to said patient an effective amount of a compound having the formula LiCl*compound I or a pharmaceutical composition comprising a compound having the formula LiCl*compound I.

### 2. Definitions

As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”. Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

In certain embodiments, the present invention provides compound I:

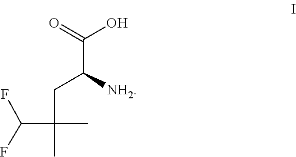

as a lithium chloride salt or co-crystal thereof.

In certain embodiments, the present invention provides compound I:

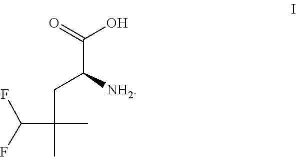

as a lithium chloride co-crystal thereof.

### 4. Uses, Formulation and Administration

### Pharmaceutically Acceptable Compositions

According to another embodiment, the invention provides a composition comprising compound I and lithium chloride and a pharmaceutically acceptable carrier, adjuvant, or vehicle. According to another embodiment, the invention provides a composition comprising compound I as a lithium chloride salt or co-crystal and a pharmaceutically acceptable carrier, adjuvant, or vehicle.

The amount of compound I and lithium chloride in compositions of this invention is such that is effective to measurably modulate or activate mTORC1, in a biological sample or in a patient. In certain embodiments, the amount of compound I and lithium chloride in compositions of this invention is such that is effective to measurably modulate or activate mTORC1, in a biological sample or in a patient. In certain embodiments, compound I and the lithium chloride form a co-crystal.

In certain embodiments, a composition of this invention is formulated for administration to a patient in need of such composition. In some embodiments, a composition of this invention is formulated for oral administration to a patient.

The term “patient,” as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of a composition comprising compound I and lithium chloride include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this invention are formulated for oral administration. Such formulations may be administered with or without food. In some embodiments, pharmaceutically acceptable compositions of this invention are administered without food. In other embodiments, pharmaceutically acceptable compositions of this invention are administered with food.

The amount of a composition comprising compound I and lithium chloride that may be combined with the carrier materials to produce a composition in a single dosage form will vary depending upon the host treated, the particular mode of administration. Preferably, provided compositions should be formulated so that a dosage of between 0.01-100 mg/kg body weight/day of the inhibitor can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a composition comprising compound I and a lithium chloride salt in the composition will also depend upon the particular compound in the composition.

### Uses and Pharmaceutically Acceptable Compositions

A composition comprising compound I and lithium chloride is generally useful for the modulation or activation of mTORC1. In some embodiments, compound I as a lithium chloride salt or co-crystal, or composition thereof, is a modulator of mTORC1. In some embodiments, compound I as a lithium chloride salt or co-crystal, or composition thereof, is a selective modulator of mTORC1. In some embodiments, compound I as a lithium chloride salt or co-crystal, or composition thereof, is an activator of mTORC1.

The activity of a composition comprising compound I and lithium chloride as a modulator or activator of mTORC1, may be assayed in vitro, in vivo or in a cell line. In vitro assays include assays that determine the modulation or activation of mTORC1. Detailed conditions for assaying a compound utilized in this invention as modulator or activator of mTORC1, are set forth in the Examples below.

As used herein, the terms “treatment.” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

A composition comprising compound I and lithium chloride is a modulator or activator of mTORC1 and is therefore useful for treating one or more disorders associated with activity of mTORC1. Thus, in certain embodiments, the present invention provides a method for treating an mTORC1-mediated disorder comprising the step of administering to a patient in need thereof a composition comprising compound I and lithium chloride, or pharmaceutically acceptable composition thereof.

As used herein, the terms “mTORC1-mediated” disorders, diseases, and/or conditions as used herein means any disease or other deleterious condition in which mTORC1, is known to play a role. Accordingly, another embodiment of the present invention relates to treating or lessening the severity of one or more diseases in which mTORC1 is known to play a role.

In some embodiments, the method of activating mTORC is used to treat or prevent depression. (See Ignicio et al., (2015) Br J Clin Pharmacol. November 27). Accordingly, in some embodiments, the present invention provides a method of treating or preventing depression, in a patient in need thereof, comprising the step of administering to said patient compound I as a lithium chloride salt or co-crystal or pharmaceutically acceptable composition thereof. In some embodiments, the depression is major depressive disorder (“MDD”). Accordingly, in some embodiments, the present invention provides a method of treating or preventing major depressive disorder, in a patient in need thereof, comprising the step of administering to said patient compound I as a lithium chloride salt or co-crystal or pharmaceutically acceptable composition thereof. In some embodiments, the depression is treatment-resistant depression (“TRD”). Accordingly, in some embodiments, the present invention provides a method of treating or preventing treatment-resistant depression, in a patient in need thereof, comprising the step of administering to said patient compound I as a lithium chloride salt or co-crystal or pharmaceutically acceptable composition thereof. In some embodiments, the treatment resistant depression is resistant to first line treatments. In some embodiments, the treatment resistant depression is resistant to second line treatments.

In some embodiments, the present invention provides a method of treating depression in a patient in need thereof, wherein said patient experiences a 50% reduction in depression scale score. In some embodiments, the patient experiences a 50% reduction in depression scale score within fewer than six weeks of administration of the compound or pharmaceutically acceptable composition. In some embodiments, the patient experiences a 50% reduction in depression scale score within fewer than four weeks of administration of the compound or pharmaceutically acceptable composition. In some embodiments, the patient experiences a 50% reduction in depression scale score within two weeks of administration of the compound or pharmaceutically acceptable composition. In some embodiments, the patient experiences a 50% reduction in depression scale score within fewer than two weeks of administration of the compound or pharmaceutically acceptable composition. In some embodiments, the patient experiences a 50% reduction in depression scale score within one week of administration of the compound or pharmaceutically acceptable composition. In some embodiments, the patient experiences a 50% reduction in depression scale score within seven days of administration of the compound or pharmaceutically acceptable composition. In some embodiments, the patient experiences a 50% reduction in depression scale score within six days of administration of the compound or pharmaceutically acceptable composition. In some embodiments, the patient experiences a 50% reduction in depression scale score within five days of administration of the compound or pharmaceutically acceptable composition. In some embodiments, the patient experiences a 50% reduction in depression scale score within four days of administration of the compound or pharmaceutically acceptable composition. In some embodiments, the patient experiences a 50% reduction in depression scale score within three days of administration of the compound or pharmaceutically acceptable composition. In some embodiments, the patient experiences a 50% reduction in depression scale score within two days of administration of the compound or pharmaceutically acceptable composition. In some embodiments, the patient experiences a 50% reduction in depression scale score within one day of administration of the compound or pharmaceutically acceptable composition. In some embodiments, the patient experiences a 50% reduction in depression scale score within twenty-four hours of administration of the compound or pharmaceutically acceptable composition. In some embodiments, the depression scale score is selected from the Montgomery-Asberg Depression Rating Scale (MADRS), the Hamilton Depression Rating Scale (HAMD-6), the Inventory of Depression Symptomatology Self-Rated Scale (IDS-SR), and the Clinical Global Impression Severity Scale (CGI-S).

In some embodiments, the present invention provides a method of treating depression in a patient in need thereof, comprising the step of orally administering to said patient compound I as a lithium chloride salt or co-crystal or pharmaceutically acceptable composition thereof, wherein the patient experiences a reduction in depression scale score comparable to ketamine administered via i.p. injection. In some embodiments, the reduction in depression scale score results from a single oral administration. In some embodiment, the reduction in depression scale score results from a plurality of oral administrations.

In some embodiments, the method of activating mTORC1 is used to elicit a rapid onset antidepressant activity. Accordingly, in some embodiments, the present invention provides a method of eliciting a rapid onset antidepressant activity, in a patient in need thereof suffering from TRD, comprising the step of administering to said patient compound I as a lithium chloride salt or co-crystal or pharmaceutically acceptable composition thereof. In some embodiments, the rapid onset antidepressant activity occurs within two weeks of administration of said compound or composition. In some embodiments, the rapid onset antidepressant activity occurs within one week of administration of said compound or composition. In some embodiments, the rapid onset antidepressant activity occurs within seven days of administration of said compound or composition. In some embodiments, the rapid onset antidepressant activity occurs within six days of administration of said compound or composition. In some embodiments, the rapid onset antidepressant activity occurs within five days of administration of said compound or composition. In some embodiments, the rapid onset antidepressant activity occurs within four days of administration of said compound or composition. In some embodiments, the rapid onset antidepressant activity occurs within three days of administration of said compound or composition. In some embodiments, the rapid onset antidepressant activity occurs within two days of administration of said compound or composition. In some embodiments, the rapid onset antidepressant activity occurs within one day of administration of said compound or composition. In some embodiments, the rapid onset antidepressant activity occurs within less than twenty-four hours of administration of said compound or composition.

In some embodiments, the present invention provides a method of eliciting a long-lasting, sustained antidepressant activity, in a patient in need thereof suffering from depression, comprising the step of administering to said patient compound I as a lithium chloride salt or co-crystal or pharmaceutically acceptable composition thereof. In some embodiments, the patient in need suffers from TRD. In some embodiments, the long-lasting, sustained antidepressant activity persists for at least twenty-four hours after a single administration of compound I as a lithium chloride salt or co-crystal or a pharmaceutically acceptable composition thereof. In some embodiments, the long-lasting, sustained antidepressant activity persists for longer than one day. In some embodiments, the long-lasting, sustained antidepressant activity persists for at least two days. In some embodiments, the long-lasting, sustained antidepressant activity persists for at least three days. In some embodiments, the long-lasting, sustained antidepressant activity persists for at least four days. In some embodiments, the long-lasting, sustained antidepressant activity persists for at least five days. In some embodiments, the long-lasting, sustained antidepressant activity persists for at least six days. In some embodiments, the long-lasting, sustained antidepressant activity persists for at least seven days.

In some embodiments, the present invention provides a method of eliciting antidepressant activity that is both rapid onset and long-lasting, sustained.

In some embodiments, the present invention provides a method of eliciting a positive behavioral response in a subject, comprising the step of administering to said subject compound I as a lithium chloride salt or co-crystal or pharmaceutically acceptable composition thereof. In some embodiments, the positive behavioral response correlates with an improvement in mood. In some embodiments, the positive behavioral response correlates with an reduction of anxiety. In some embodiments, the positive behavioral response corresponds with an improvement in mood. In some embodiments, the positive behavioral response correlates with an improved ability to cope with stress.

In some embodiments, the present invention provides a method of eliciting a rapid onset, positive behavioral response is a subject, comprising the step of administering to said subject compound I as a lithium chloride salt or co-crystal or pharmaceutically acceptable composition thereof. In some embodiments, the positive behavioral response occurs within twenty-four hours of administration. In some embodiments, the positive behavioral response occurs within one day of administration. In some embodiments, the positive behavioral response occurs within two days of administration. In some embodiments, the positive behavioral response occurs within three days of administration. In some embodiments, the positive behavioral response occurs within four days of administration. In some embodiments, the positive behavioral response occurs within five days of administration. In some embodiments, the positive behavioral response occurs within six days of administration. In some embodiments, the positive behavioral response occurs within seven days of administration. In some embodiments, the positive behavioral response occurs within one week of administration.

In some embodiments, the present invention provides a method of eliciting a long-lasting, sustained positive behavioral response in a subject, comprising the step of administering to said patient compound I as a lithium chloride salt or co-crystal or pharmaceutically acceptable composition thereof. In some embodiments, the long-lasting, sustained positive behavioral response persists for longer than one day. In some embodiments, the long-lasting, sustained positive behavioral response persists for at least two days. In some embodiments, the long-lasting, sustained positive behavioral response persists for at least three days. In some embodiments, the long-lasting, sustained positive behavioral response persists for at least four days. In some embodiments, the long-lasting, sustained positive behavioral response persists for at least five days. In some embodiments, the long-lasting, sustained positive behavioral response persists for at least six days. In some embodiments, the long-lasting, sustained positive behavioral response persists for at least seven days.

In some embodiments, the present invention provides a method of eliciting a positive behavioral response that is both rapid onset and long-lasting, sustained.

In some embodiments, the present invention provides a method of ameliorating and/or reversing behavioral and synaptic defects caused by chronic, unpredictable stress (CUS), in a patient in need thereof, comprising the step of administering to said patient compound I as a lithium chloride salt or co-crystal or pharmaceutically acceptable composition thereof. In some embodiments, the method ameliorates and/or reverses behavioral defects caused by CUS. In some embodiments, the method ameliorates and/or reverses synaptic defects caused by CUS. In some embodiments, the synaptic defect caused by CUS is a decrease in postsynaptic protein expression. In some embodiments, the decrease in postsynaptic protein expression is a decrease in the expression of GLUR1 or PSD95.

In some embodiments, the method of activating mTORC1 is used to treat or prevent forms of autism. (See Novarino et al., (2012) Science 19 October, 338:6105, pp. 394-397). Accordingly, in some embodiments, the present invention provides a method of treating or preventing a form of autism, in a subject in need thereof, comprising the step of administering to said subject compound I as a lithium chloride salt or co-crystal or pharmaceutically acceptable composition thereof. In some embodiments, the autism is a genetic form of autism.

In some embodiments, the present invention provides a method of treating a genetic form of autism in a patient in need thereof comprising the step of administering to said patient compound I as a lithium chloride salt or co-crystal or pharmaceutically acceptable composition thereof. SHANK3 haploinsufficiency is causative for the neurological features of Phelan-McDermid syndrome (PMDS), including a high risk of autism spectrum disorder (Bidinosti et al. (2016) Science Reports 351, 1199-1203). Down regulation of mTORC1 in SHANK3 deficient neurons is due to enhanced phosphorylation and activation of serine/threonine protein phosphatase 2A (PP2A) regulatory subunit, B56b, by its kinase, Cdc2-like kinase 2 (Bidinosti et al. (2016) Science Reports 351, 1199-1203). SHANK3 mutant mice show autistic traits (Yang et al. (2012) The Journal of Neuroscience 32, 6525-6541). Patients with autistic traits and motor delays carry deleterious homozygous mutations in the SLC7A5 gene. Solute carrier transporter 7a5 (SLC7A5), a large neutral amino acid transporter localized at the blood brain barrier (BBB), has an essential role in maintaining normal levels of brain BCAAs. Leucine intracerebroventricular administration ameliorates abnormal behaviors in adult mutant mice (Tarlungeanu et al. (2016) Cell 167, 1481-1494).

In some embodiments, the present invention provides a method of treating a Lysosomal Storage Disease or Disorder (“LSD”) in a patient in need thereof, comprising the step of administering to said patient compound I as a lithium chloride salt or co-crystal or pharmaceutically acceptable composition thereof. LSDs are a group of inherited metabolic disorders that result from defects in lysosomal function. Lysosomal storage disorders are caused by lysosomal dysfunction usually as a consequence of deficiency of a single enzyme required for the metabolism of lipids, glycoproteins (sugar containing proteins) or so-called mucopolysaccharides. In some embodiments, the present invention provides a method of treating a lipid storage disorder in a patient in need thereof, comprising the step of administering to said patient compound I as a lithium chloride salt or co-crystal or pharmaceutically acceptable composition thereof. In some embodiments, the lipid storage disorder is selected from a sphingolipidose (e.g., gangliosidosis, Gaucher, Niemann-Pick disease, or Metachromatic leukodystrophy). In some embodiments, the present invention provides a method of treating a gangliosidosis (e.g., Tay-Sachs disease or a leukodystrophy). In some embodiment, the present invention provides a method of treating a mucopolysaccharidoses in a patient in need thereof, comprising the step of administering to said patient compound I as a lithium chloride salt or co-crystal or pharmaceutically acceptable composition thereof. In some embodiments, the mucopolysaccharidoses is Hunter syndrome or Hurler disease.

In some embodiments, the present invention provides a method of treating JNCL (Batten Disease) in a patient in need thereof, comprising the step of administering to said patient compound I as a lithium chloride salt or co-crystal or pharmaceutically acceptable composition thereof. JNCL is caused by the deletion of exons 7 and 8 of the CLN3 gene resulting in a nonfunctional protein. Battenin, the full-length protein encoded by CLN3, is a transmembrane protein that localizes to the late endosome and lysosome where it has been shown to help regulate pH, amino acid balance and vesicle trafficking (Pearce et al. (1999) Nature Genetics 22, 1; Fossale et al. (2004) BMC Neuroscience 10, 5) mTOR activation requires intracellular nutrients provided by autophagy which, in in vitro and in vivo models of JNCL, are lowered due to the lack of functional battenin (Cao et al. (2006) Journal of Biological Chemistry 281, 29).

In some embodiments, the present invention provides a method of treating cystinosis in a patient in need thereof, comprising the step of administering to said patient compound I as a lithium chloride salt or co-crystal or pharmaceutically acceptable composition thereof. Cystinosis is an autosomal recessive disease affecting those with two alleles mutated in the CSTN gene; the lysosomal cystine transporter cystinosin is defective in efflux of cystine from the lysosome, resulting in cystine crystal formation in renal epithelial tubules and loss of kidney function. Studies have shown defective or reduced mTORC1 signaling in cells lacking CSTN and mislocalized mTOR (Ivanova et al. (2016) J Inherit Metab Dis. 39(3), 457-64; Andrzejewska et al. (2016) J Am Soc Nephrol. 27(6), 1678-1688e). These defects could not be rescued by cysteamine (Ivanova et al. (2016) J Inherit Metab Dis. 39(3), 457-64; Andrzejewska et al. (2016) J Am Soc Nephrol. 27(6), 1678-1688e). Cystinosin has also been found to bind mTORC1 pathway components v-ATPase, Rags, and Ragulator (Andrzejewska et al. (2016) J Am Soc Nephrol. 27(6), 1678-1688e). CTNS-deficient cells show and increased number of autophagosomes and reduced chaperone-mediated autophagy (Napolitano et al. (2015) EMBO Mol Med. 7(2), 158-74).

In some embodiments, the present invention provides a method of treating Fabry disease in a patient in need thereof, comprising the step of administering to said patient compound I as a lithium chloride salt or co-crystal or pharmaceutically acceptable composition thereof. In Fabry disease, deficiencies in alpha-galactosidase results in the lysosomal accumulation of globotriaosylceramide lipids. Decreased mTOR activity and increased autophagy is observed in vitro and in vivo in a cell model of Fabry in which alpha-galactosidase is knocked down with shRNA (Liebau et al. (2013) PLoS 8, e63506). Hyperactive autophagy is also observed in the brains of mice in which alpha-galactosidase is knocked out (Nelson et al. (2014) Acta Neuropathologica Communications 2, 20).

In some embodiments, the present invention provides a method of treating Mucolipidosis type IV (MLIV) in a patient in need thereof, comprising the step of administering to said patient compound I as a lithium chloride salt or co-crystal or pharmaceutically acceptable composition thereof. In MLIV, mutations of the TRPML1 lysosomal Ca(2+) channel cause disordered lysosomal membrane trafficking. An MLIV knockout in Drosophila resulted in upregulation of autophagy and a decrease in mTOR activity, both of which could be reversed by activating mTORC1 genetically or by feeding animals a high protein diet (Wong et al. (2012) Curr Biol. 22(17), 1616-1621). Increased autophagy is also observed in fibroblasts from MLIV patients (Vergarajauregui et al. (2008) Human Molecular Genetics 17, 2723-2737).

In some embodiments, the present invention provides a method of treating mental retardation in a patient in need thereof, comprising the step of administering to said patient compound I as a lithium chloride salt or co-crystal or pharmaceutically acceptable composition thereof. In Homo sapiens, Cereblon mutations are linked to a mild form of autosomal recessive non-syndromic mental retardation. In a mouse cereblon knockout model of retardation, loss of cereblon activates AMPK, inhibits mTOR and reduces protein translation in the cerebellum (Lee et al. (2014) J Biol Chem. 289, 23343-52; Xu et al. (2013) J Biol Chem. 288, 29573-85).

In some embodiments, the present invention provides a method of increasing neuronal protein expression in a subject, comprising the step of administering to said subject compound I as a lithium chloride salt or co-crystal or pharmaceutically acceptable composition thereof. In some embodiments the increase in neuronal protein expression occurs in a postsynaptic neuron. In some embodiments, the increase in neuronal protein expression includes increased expression of brain-derived neurotrophic factor (BDNF). In some embodiments, the increase in neuronal protein expression includes increased expression of glutamate receptor 1 (GluR1). In some embodiments, the increase in neuronal protein expression includes increased expression of synapsin. In some embodiments, the increase in neuronal protein expression includes increased expression of PSD95.

In some embodiments, the present invention provides a method of increasing synaptogenesis in a subject, comprising the step of administering to said subject compound I as a lithium chloride salt or co-crystal or pharmaceutically acceptable composition thereof. In some embodiments, the increased synaptogenesis involves synaptic remodeling. In some embodiments, the increased synaptogenesis involves induction of dendritic spines. In some embodiments, the induction of dendritic spines causes an increased density of dendritic spines. In some embodiments, the dendritic spines are thin spines. In some embodiments, the dendritic spines are mushroom spines.

In some embodiments, the present invention provides a method of enhancing synoptic function in a subject, comprising the step of administering to said subject compound I as a lithium chloride salt or co-crystal or pharmaceutically acceptable composition thereof. In some embodiments, the enhanced synoptic function in a subject involves an increase in excitatory postsynaptic currents (EPSC).

In some embodiments, the present invention provides a method of treating disorders of the central nervous system (CNS), comprising the step of administering to said patient compound I as a lithium chloride salt or co-crystal or pharmaceutically acceptable composition thereof. In some embodiments, the CNS disorder is bipolar disorder, depression or schizophrenia.

In some embodiments, the present invention provides a method of treating an eating disorder, comprising the step of administering to said patient compound I as a lithium chloride salt or co-crystal or pharmaceutically acceptable composition thereof. In some embodiments, the eating disorder is anorexia or bulimia.

In some embodiments, the present invention provides a method of treating a blood disorder, comprising the step of administering to said patient compound I as a lithium chloride salt or co-crystal or pharmaceutically acceptable composition thereof. In some embodiments, the blood disorder is anemia and low white-cell count (neutropenia).

In some embodiments, the present invention provides a method of treating headache, alcoholism, post-traumatic stress disorder (PTSD), epilepsy, diabetes, liver disease, kidney disorders, arthritis, skin conditions such as seborrhea, overactive thyroid, asthma, Huntington's disease, Graves' disease, herpes simplex, movement disorders such as tardive dyskinesia, Tourette's syndrome, cyclical vomiting, Meniere's disease, tingling or “crawling” sensation in the skin (paresthesias), and aggressive behavior in attention deficit-hyperactivity disorder (ADHD), comprising the step of administering to said patient compound I as a lithium chloride salt or co-crystal or pharmaceutically acceptable composition thereof.

Pharmaceutically acceptable compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. In certain embodiments, compound I as a lithium chloride salt or co-crystal may be administered orally or parenterally at dosage levels of about 0.01 mg/kg to about 200 mg/kg, or dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of compound I as a lithium chloride salt or co-crystal, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing compound I as a lithium chloride salt or co-crystal with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of compound I as a lithium chloride salt or co-crystal include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

According to one embodiment, the invention relates to a method of modulating mTORC1 activity in a biological sample comprising the step of contacting said biological sample with compound I as a lithium chloride salt or co-crystal, or a composition comprising said compound.

According to one embodiment, the invention relates to a method of selectively modulating mTORC1 activity in a biological sample comprising the step of contacting said biological sample with compound I as a lithium chloride salt or co-crystal, or a composition comprising said compound.

According to one embodiment, the invention relates to a method of activating mTORC1 in a biological sample comprising the step of contacting said biological sample with compound I as a lithium chloride salt or co-crystal, or a composition comprising said compound.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof.

Another embodiment of the present invention relates to a method of modulating mTORC1 activity in a patient comprising the step of administering to said patient compound I as a lithium chloride salt or co-crystal, or a composition comprising said compound.

Another embodiment of the present invention relates to a method of selectively modulating mTORC1 activity in a patient comprising the step of administering to said patient compound I as a lithium chloride salt or co-crystal, or a composition comprising said compound.

Another embodiment of the present invention relates to a method of activating mTORC1 in a patient comprising the step of administering to said patient compound I as a lithium chloride salt or co-crystal, or a composition comprising said compound.

In other embodiments, the present invention provides a method for treating a disorder mediated by mTORC1 in a patient in need thereof, comprising the step of administering to said patient compound I as a lithium chloride salt or co-crystal or pharmaceutically acceptable composition thereof. Such disorders are described in detail herein.

Depending upon the particular condition, or disease, to be treated, additional therapeutic agents that are normally administered to treat that condition, may also be present in the compositions of this invention. As used herein, additional therapeutic agents that are normally administered to treat a particular disease, or condition, are known as “appropriate for the disease, or condition, being treated.”

In some embodiments, compound I as a lithium chloride salt or co-crystal is administered in combination with an antidepressant therapeutic agent. Antidepressant therapeutic agents are well known to one of ordinary skill in the art and include Selective Serotonin Reuptake Inhibitors (“SSRI”, e.g. sertraline, escitalopram, citalopram, fluvoxamine, fluoxetine, paroxetine), antidepressant (e.g., bupropion, venlafaxine, mirtazapine, duloxetine, amitriptyline, imipramine, selegiline, nortriptyline, trazodone, desvenlafaxine, and aripiprazole).

In some embodiments, compound I as a lithium chloride salt or co-crystal is administered in combination with an additional therapeutic agent or process useful for treating one or more LSDs. In some embodiments, compound I as a lithium chloride salt or co-crystal is administered in combination with enzyme replacement therapy, chemical chaperone therapy, bone marrow transplantation, substrate reduction therapy, α-L-iduronidase, Recombinant human N-acetylgalactosamine-4-sulphatase (arylsulphatase B), an inhibitor of glycosphingolipid biosynthesis, N-butyldeoxynojirimycin (Miglustat), a hydrophobic iminosugar, or an inhibitor of α-galactosidase A (e.g., 1-deoxy-galactonojirimycin).

Those additional agents may be administered separately from an inventive compound-containing composition, as part of a multiple dosage regimen. Alternatively, those agents may be part of a single dosage form, mixed together with compound I as a lithium chloride salt or co-crystal in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents in accordance with this invention. For example, compound I as a lithium chloride salt or co-crystal may be administered with another therapeutic agent simultaneously or sequentially in separate unit dosage forms or together in a single unit dosage form. Accordingly, the present invention provides a single unit dosage form comprising compound I as a lithium chloride salt or co-crystal, an additional therapeutic agent, and a pharmaceutically acceptable carrier, adjuvant, or vehicle.

The amount of both compound I as a lithium chloride salt or co-crystal and additional therapeutic agent (in those compositions which comprise an additional therapeutic agent as described above) that may be combined with the carrier materials to produce a single dosage form will vary depending upon the host treated and the particular mode of administration. Preferably, compositions of this invention should be formulated so that a dosage of between 0.01-100 mg/kg body weight/day of compound I as a lithium chloride salt or co-crystal can be administered.

In those compositions which comprise an additional therapeutic agent, that additional therapeutic agent and compound I as a lithium chloride salt or co-crystal may act synergistically. Therefore, the amount of additional therapeutic agent in such compositions will be less than that required in a monotherapy utilizing only that therapeutic agent. In such compositions a dosage of between 0.01-1,000 g/kg body weight/day of the additional therapeutic agent can be administered.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

Compound I as a lithium chloride salt or co-crystal, or pharmaceutical compositions thereof, may also be incorporated into compositions for coating an implantable medical device, such as prostheses, artificial valves, vascular grafts, stents and catheters. Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition comprising a kinase inhibitor. Implantable devices coated with compound I as a lithium chloride salt or co-crystal are another embodiment of the present invention.

All features of each of the aspects of the invention apply to all other aspects mutatis mutandis.

In order that the invention described herein may be more fully understood, the following examples are set forth. It should be understood that these examples are for illustrative purposes only and are not to be construed as limiting this invention in any manner.

### EXEMPLIFICATION

As depicted in the Examples below, in certain exemplary embodiments, compound I as a lithium chloride salt or co-crystal is prepared according to the following general procedures. It will be appreciated that, although the general methods depict the synthesis of a certain compound of the present invention, the following general methods, and other methods known to one of ordinary skill in the art, can be applied, as described herein.

### General Procedures for Compound Preparation

Compound I was prepared according to the methods described in U.S. Pat. No. 10,100,066, the entirety of which is incorporated herein by reference.

Preparation of compound I as a lithium chloride co-crystal is described below.

### Example 1: Compound I as a Lithium Chloride Salt or Co-Crystal

Two (2) g of compound 1 (11.04 mmol) and 3.51 g of lithium chloride (82.8 mmol, 7.5 equivalent) were dissolved in 250 ml (125 vol) of water to give a colorless solution. The resulting solution was concentrated to 10 vol at atmospheric pressure for approximately 5 hours. The solution was then cooled down to room temperature over a period of 1.5 hours. During the cooling process a precipitate was observed. The solid material was isolated by filtration and drying overnight under vacuum to yield a white solid (950 mg: 38.5% yield). The solid was characterized as compound I as a lithium chloride co-crystal using different techniques and the results are summarized in Table 1, below.

TABLE 1
Characterization Techniques

| Test | Result |
|---|---|
| NMR | Concordant with structure of |
| | compound I |
| ICP (Li content) | 2.84% w/w |
| TGA | Weight loss #1 11.2% w/w |
| Chloride | 14.17% w/w |
| HPLC | 89.94% w/w |
| Assay (as compound I*LiCl) | ee >99% |
| Enantiomeric purity | |

Stoichiometry based on HPLC assay, ICP, and Chloride reveal a 1:1 ratio between compound I and LiCl, as summarized in Table 2, below.

TABLE 2
Stoichiometry Data

| | Mol. Weight | | |
|---|---|---|---|
| | (g/mol) | w/w % | Moles |
| Co-Crystal | 223.577 | 89.94 | 0.402 |
| Compound I | 181.183 | | 0.402 |
| Li⁺ | 6.941 | 2.84 | 0.409 |
| Cl⁻ | 35.453 | 14.17 | 0.4 |

### General Procedures for In Vivo Tests

Animal Use: Male Sprague Dawley rats weighing 175-200 g (Charles River Laboratories. Wilmington, MA) will be group-housed upon arrival (Yale University, New Haven CT) and acclimated 5 days before initiating experimental studies. Rats will be provided food and water ad libitum except during protocol-specified fasting. The animals will be monitored daily for clinical signs. A qualified veterinarian will provide oversight for all rodent procedures. All personnel will receive training from Yale animal care and use committee (IACUC). All animal procedures will be conducted at Yale University in strict accordance with the National Institutes of Health IACUC and will be approved by the Yale Animal Care and Use Committee.

Behavioral Analysis Using the Female Urine Sniffing Test (FUST): The FUST will be conducted according to published procedures (Malkesman. O. et al., Biol Psychiatry 67(9): 864-71 (2010)), 24 hr post-dosing. Briefly, rats will be habituated for 60 min to a cotton swab dipped in tap water in their home cage. Next, rats will be exposed to a 2nd cotton swab dipped in tap water and 45 min later they will be exposed to a 3rd cotton swab infused with fresh rat urine from 11 to 14-week-old female rats in estrus. The total time (s) spent sniffing the cotton-tipped applicator will be quantitated over 5 min for each animal.

Behavioral Analysis Using the Locomotor Activity Assessment (LMA): LMA will be assessed according to published procedures (Wamer-Schmidt. J. L. & and Duman. R. S. PNAS 104(11): 4647-52 (2007)), in an open field fitted with automated activity meters consisting of parallel rows of infrared beams. The number of beam-breaks will be recorded over a 30 min interval for each animal.

Behavioral Analysis Using the Novelty Suppressed Feeding Test (NSFT): The NSFT will be conducted as previously described (Wamer-Schmidt, J. L. & Duman, R. S. PNAS 104(11): 4647-52 (2007)). Rats will be fasted in their home cages for 20 hr and then placed in a Plexiglas open field (76.5 cm×76.5 cm×40 cm) with a small amount of food in the center. Animals will be allowed to explore the open field for 8 min and the latency to feed (s) was recorded.

Behavioral Analysis Using the Sucrose Preference Test (SPT): Rats will be habituated to a palatable sucrose 1% sucrose solution for 48 hrs to avoid neophobia. Rats will be treated with compound I as a lithium chloride salt or co-crystal or Veh at the end of day 0 and the SPT will be performed 24 hrs post-administration on day 1. For the SPT, rats will be deprived of water for 6 hrs and exposed for 60 min to two bottles with equal volumes of 1% sucrose or water. The ratio of the volume of sucrose water consumed to total water consumed during the 1 hr test will be defined as sucrose preference (e.g., a ratio of 1 would indicate the rat consumed only 1% sucrose, while a ratio of 0.5 would indicate that the rat drank equal amounts of 1% sucrose and water).

Chronic Unpredictable Stress (CUS) Conditions: Rats will be exposed to a variable sequence of 12 unpredictable stressors, preventing habituation as described (Li, N. et al., Biol Psychiatry 69(8): 754-61 (2011)). The following twelve stressors will be applied (2 per day, for 25 days): cage rotation, light on, light off, cold stress, isolation, swim stress, food and water deprivation, wet bedding, stroboscope, cage tilt, odor exposure and group housing. The animals in non-stressed (NS) groups will be housed normally without the application of external stressors. Both NS and CUS rats will be handled and weighed weekly.

Marmoset Human Threat Test (HTT): Marmosets will be challenged by the presence of a human observer over a long period of time on a regular basis. Such chronic stimulation is known to increase plasma cortisol and the subsequent increase in hypothalamic-pituitary-adrenal function contributed to the pathophysiology of depressive illness.

### Example A: Behavioral Changes in the Novelty Suppressed Feeding and Female Urine Sniffing Tests after a Single Dose of Compound I as a Lithium Chloride Salt or Co-Crystal or Ketamine

Study Design: Male Sprague Dawley Male rats weighing between 175 and 200 g will be randomized into four (4) study groups, following a 5-day acclimation period. On study day 0, rats will receive a single dose of either saline (Sal) or ketamine (Ket) in Groups 1 and 2, respectively, by intraperitoneal injection (i.p.). The rats in Group 3 and 4 will receive a single dose of the compound I vehicle (Veh, 0.5% Methylcellulose/0.1% Tween-80) or compound I as a lithium chloride salt or co-crystal (160 mg/kg), respectively, by oral gavage. All rats will be subjected to the FUST on day 1, 24 hrs after dosing. On day 2, 48 hrs after dosing, the LMA of all rats will be measured in an open field. Rats will then be fasted for 20 hrs and subjected to the NSFT, 72 hrs post-dose.

Preparation of Test Articles: Ket (Sigma, cat #K1884) will be dissolved in Sal at a concentration of 10 mg/mL. A volume of 1 ml/kg of Sal or Ket will be injected i.p. for Groups 1 and 2, respectively. Compound I as a lithium chloride salt or co-crystal will be prepared by dissolving in Veh (0.5% methylcellulose/0.1% Tween-80) at a concentration of 50 mg/mL. The dosing volume based on the weight of the animal (3.2 mL/kg) of Veh or compound I as a lithium chloride salt or co-crystal will be administered by oral gavage to study animals in Groups 3 and 4, respectively. Test articles will be prepared the day of dosing.

### Example B: Comparative Effects of Single Dose of Compound I as a Lithium Chloride Salt or Co-Crystal and Ketamine Administration of the mTORC1 Signaling Pathway and Synaptic Protein Expression in Synaptosome Preparations Derived from the Rat Pre-Frontal Cortex

Study Design: Male Sprague Dawley rats weighing between 175 and 200 g will be randomized into eight (8) study groups, following a 5-day acclimation period. On study day 0, rats in Groups 3 and 7 will receive a single dose of Sal while Groups 4 and 8 will receive a single dose of Ket (10 mg/kg) each by i.p. injection. The rats in Groups 1 and 5 will received a single dose of Veh while Groups 2 and 6 will receive a single dose of compound I as a lithium chloride salt or co-crystal (160 mg/kg) each by oral gavage. One hour after dosing, rats in Groups 1-4 will be sacrificed by conscious decapitation and followed by collection of the PFC. Crude synaptosomes will be prepared from the PFC and three mTORC1 substrates, pmTOR, pp70S6K and p4E-BP1, and corresponding total protein loading controls (mTOR, p70S6K, and GAPDH) will be quantitated by Western blot. Twenty-four hours after dosing, rats in Groups 5-8 will be sacrificed by conscious decapitation and collection of the PFC. Crude synaptosomes will be prepared from the PFC and the synaptic proteins (GluR1 and PSD95), as well as the total protein loading control (GAPDH), will be quantitated by Western blot.

Formulation of Ket and Compound I as a Lithium Chloride Salt or Co-Crystal for Administration: Ket (Sigma, cat #K1884) will be dissolved in Sal at a concentration of 10 mg/mL. A volume of 1 mL/kg will be injected i.p. Compound I as a lithium chloride salt or co-crystal will be prepared by dissolving in Veh at a concentration of 50 mg/mL. The dosing volume based on the weight of the animal (3.2 mL/kg) will be administered by oral gavage. Test articles will be prepared the day of dosing.

Prefrontal Cortex Synaptosome Preparations: The brain will be dissected from rats in all groups and rinsed in PBS. The PFC will be collected and homogenized at 4° C. in homogenization buffer (0.32 M sucrose. 20 mM HEPES at pH 7.4, 1 mM EDTA, 5 mM NaF, 1 mM NaVO₃ and protease inhibitor cocktail (Roche: #19543200)). The homogenate will be centrifuged for 10 min at 2,800 rpm at 4° C. after which the supernatant will be removed and re-centrifuged at 12,000 rpm for 10 min at 4° C. The resulting pellet containing crude synaptosomes will be re-suspended in Lysis buffer (50 mM Tris-HCl (pH 7.5), 150 mM NaCl, 1% Triton X-100, 0.1% SDS, 2 mM, EDTA, 1 mM NaVO₃, 5 mM NaF and protease inhibitor cocktail) and sonicated on ice for 20 s at 50% amplitude. The protein concentration will be determined by Bradford assay and all samples will be mixed with loading buffer (60 mM Tris-HCl pH 6.8, 20 mM DTT, 2% SDS, 10% glycerol, 5% β-mercaptoethanol and 0.01% Bromophenol blue) and stored at −20° C. until WB analysis.

Western Blot Analysis: Western blot analysis for GluR1, PSD95 and GAPDH will be performed as previously described. Briefly, synaptosomes preparations (15 μg total protein) will be loaded into 10-15% SDS PAGE gel for electrophoresis and transferred to a polyvinylidene difluoride (PVDF) membrane in transfer buffer (10× premixed electrophoresis buffer contains 25 mM Tris. 192 mM glycine. pH 8.3; Bio-Rad). The PVDF membranes will be blocked for 1 hr at room temperature with blocking buffer (2% BSA in PBS-T (10 mM Phosphate, pH 7.4, 2.7 mM KCl, 137 mM NaCl and 0.1% Tween-20)) and subsequently incubated with primary antibodies: rabbit anti-pmTOR at 1:1000 (Cell Signaling; #5536), rabbit anti-mTOR at 1:1000 (Cell Signaling; #2972), rabbit anti-pp70S6K at 1:1000 (Cell Signaling; #9205), rabbit anti-p70S6K at 1:1000 (Cell Signaling; #2708), rabbit anti-p4E-BP1 at 1:1000 (Cell Signaling; #2855), rabbit anti-GluR1 at 1:1000 (Cell Signaling; #13185), rabbit anti-Synapsin 1 at 1:1000 (Cell Signaling; #5297), rabbit anti-PSD95 at 1:1000 (Cell Signaling; #9644) and rabbit anti-GAPDH at 1:1000 (Cell Signaling; #5174) in blocking buffer overnight at 4° C. The next day, membranes will be washed 3 times in PBS-T buffer and incubated with horseradish peroxidase conjugated anti-mouse or anti-rabbit secondary antibody at 1:5000 to 1:10000 (Vector Laboratories Inc) for 1 hr. After final three washes with PBS-T buffer, bands will be detected using enhanced chemiluminescence. The blots then will be incubated in the stripping buffer (2% SDS, 100 mM β-mercaptoethanol, 50 mM Tris-HCl pH 6.8) for 30 min at 50-55° C. followed by three washes with PBS-T buffer. The stripped blots will be kept in blocking solution for 1 hr and incubated with the primary antibody directed against total levels of the respective protein or GAPDH for loading control. Densitometric analysis of phospho- and total immunoreactivity for each protein will be conducted using NIH Image J software. The resulting densitometric readings will be used to generate a ratio of phopho-protein to their respective total protein levels or GAPDH as indicated. The resulting ratio will be further normalized to Sal or Veh treated control group for each protein.

### Example C: Effect of Single Roal Dose of Compound I as a Lithium Chloride Salt or Co-Crystal on mTORC1 Signaling Pathway in Multiple Regions of the Rat Brain

Study Design: Male rats weighing between 175 and 200 g will be randomized into two study groups, following a 5-day acclimation period. Group 1 will receive a single administration of Veh by oral gavage and group 2 will receive a single administration of compound I as a lithium chloride salt or co-crystal (160 mg/kg prepared in Veh) by oral gavage. One hour following administration, rats will be sacrificed by conscious decapitation, plasma will be collected for analysis of compound I as a lithium chloride salt or co-crystal exposure in addition to the isolation of the PFC, hippocampus, striatum, neocortex and cerebellum by microdissection. Total protein extracts will be prepared from the harvested tissues and submitted to WB analysis followed by quantitative analysis of selected mTORC1 substrates.

Formulation of Compound I as a Lithium Chloride Salt or Co-Crystal (160 mg/ml): Compound I as a lithium chloride salt or co-crystal will be prepared by dissolving in Veh at a concentration of 160 mg/mL. The dosing volume based on the weight of the animal (10 mL/kg) will be administered by oral gavage to study animals in Group 2. Test articles will be prepared the day of dosing.

Western Blot Analysis: Synaptosomes preparations (15 μg of total protein) will be loaded and separated on NUPAGE 4-12% Bis-Tris gels and transferred to PVDF membrane (Immobilon-FL PVDF membrane, Millipore) using CAPS Buffer (10 mM 3-(Cyclohexylamino)-1-propanesulfonic acid, 12.5% Ethanol pH=10). After transfer, membranes will be incubated for 1 hr at room temperature in Odyssey blocking buffer (Licor). After blocking, membranes will be incubated at 4° C. overnight with primary antibodies. Primary antibodies used will be rabbit anti-^{S400/440}pS6 (cell signaling; #5364) at 1:1000 and mouse anti-α-tubulin (Sigma; #T5168) at 1:10000 in Odyssey blocking buffer. The next day, membranes will be washed three times in 1×TBS-Tween (25 mM Tris, pH 7.4, 3.0 mM KCl, 140 mM NaCl and 0.05% Tween-20) and incubated with dye-coupled secondary antibodies (goat anti-mouse IRdye680, and goat anti-rabbit IRdye800 from LI-COR) at 1:20000 in Odyssey blocking buffer for 30 min then wash three times in 1×TBS-Tween. Signals will be quantified using the Odyssey Infrared Imaging System (LI-COR Bioscience). The resulting densitometric readings will be used to generate a ratio of phopho-protein to α-tubulin. The resulting ratio will be further normalized to vehicle treated control group.

Prefrontal Cortex Synaptosonie Preparations: One hour following dosing rats will be sacrificed by conscious decapitation, plasma and brain will be collected. Brains will be dissected for each group and rinsed in PBS. PFC, striatum, hippocampus, neocortex and cerebellum will be collected and homogenized at 4° C. in homogenization buffer (0.32 M sucrose, 20 mM HEPES at pH 7.4, 1 mM EDTA, 5 mM NaF, 1 mM NaVO₃ and protease inhibitor cocktail (Roche; Ser. No. 19/543,200)). Homogenates will be centrifuged for 10 min at 2,800 rpm at 4° C. after which supernatants will be removed and re-centrifuged at 12.000 rpm for 10 mm at 4° C. Resulting pellets will be re-suspended in Lysis buffer (50 mM Tris-HCl (pH 7.5), 150 mM NaCl, 1% Triton X-100, 0.1% SDS, 2 mM, EDTA, 1 mM NaVO₃, 5 mM NaF and protease inhibitor cocktail) and sonicated on ice for 20 s at 50% amplitude. Total protein concentrations will be determined by Bradford assay and all samples will be mixed with loading buffer (50 mM Tris-HCl pH 6.8, 2% SDS, 5% glycerol, 5% β-mercaptoethanol and 0.01% Bromophenol blue) and stored at −20° C. until WB analysis.

Compound Analysis: For determination of compound levels in plasma, proteins will be precipitated from 50 μL of the resulting tissue homogenate containing 150 μL of the internal standard (tolbutamide) in acetonitrile followed by centrifugation at 3000 rpm for 10 min. One hundred microliters of the resulting supernatant will be added to 100 μL of water, mixed well and injected on the LC-MS/MS system using the following program for assessing compound levels:
Phenomenex LUX Cellulose column (4.6×150 mm, 5 m)
Mobile Phase A—0.1% Formic Acid in Water
Mobile Phase B—0.1% Formic Acid in Acetonitrile
Gradient:
   Initial—40% A
   2 minutes—40% A
   2.1 minutes—2% A
   3 minutes—2% A
   3.1 minutes—40% A
   4 minutes—40% A
Flow rate 0.8 mL/min
Column Temp 40 degrees C.
Sciex 5500 Triple Quad Mass Spec

### Example D: Effect of Single Oral Dose of Compound I as a Lithium Chloride Salt or Co-Crystal or Leucine on mTORC1 Signaling Pathway in Rat Brain and Selected Peripheral Organs

Study Design: Male rats weighing between 175 and 200 g will be randomized into three (3) study groups following a 5-day acclimation period. Test articles will be administered by oral gavage. One hour following dosing rats will be sacrificed by conscious decapitation, plasma, brain and selected peripheral tissues will be harvested for compound level and Western blot analysis. Tissues will be prepared for Western blot to quantitate the mTORC1 substrate pS6 as a measure of mTORC1 activity.

Preparation of Test Articles: Compound I as a lithium chloride salt or co-crystal and Leucine (Leu, Sigma; #L8912) will be prepared by dissolving in Veh (0.5% methylcellulose/0.1% Tween-80) at a concentration of 16 mg/mL and 100 mg/mL, respectively. The dosing volume based on the weight of the animal (10 mL/kg) will be administered by oral gavage. Test articles will be prepared the day of dosing.

Tissues Preparations: One hour following dosing rats will be sacrificed by conscious decapitation, plasma, brain and peripheral tissues were harvested and immediately frozen in liquid nitrogen. Tissues will be thawed and homogenized for 1 min twice using MP homogenizer in Lysis Buffer (Cell lysis buffer: 1% Triton X-100, 50 mM HEPES pH 7.4, 100 mM NaCl, 2 mM EDTA, 10 mM Beta-glycerophosphate, 10 mM Sodium pyrophosphate, and 1 protease inhibitor tab per 50 mL fresh) at 4° C. Lysates will then be sonicated on ice for 20 s at 50% amplitude. The protein concentration will be determined by Bradford assay and all samples will be mixed with loading buffer (50 mM Tris-HCl pH 6.8, 2% SDS, 5% glycerol, 5% p3-mercaptoethanol and 0.01% Bromophenol blue) and stored at −20° C. until WB analysis.

Western Blot (WB) Analysis: Equal amounts of each sample (15 μg of total protein) will be loaded and separated on NuPAGE 4-12% Bis-Tris gels and transferred to PVDF membrane (Immobilon-FL PVDF membrane, Millipore) using CAPS Buffer (10 mM 3-(Cyclohexylamino)-1-propanesulfonic acid, 12.5% Ethanol pH=10). After transfer, membranes will be incubated for 1 hr at room temperature in Odyssey blocking buffer (Licor). After blocking, membranes will be incubated at 4° C. overnight with primary antibodies. Primary antibodies used will be rabbit anti-S400/440pS6 (cell signaling; #5364) at 1:1000, mouse anti-GAPDH (Sigma: #G8795) at 1:1000 and mouse anti-α-tubulin (Sigma; #T5168) at 1:10000 in Odyssey blocking buffer. The next day, membranes will be washed three times in 1×TBS-Tween (25 mM Tris, pH 7.4, 3.0 mM KCl, 140 mM NaCl and 0.05% Tween-20) and incubated with dye-coupled secondary antibodies (goat anti-mouse IRdye680, and goat anti-rabbit IRdye800 from LI-COR) at 1:20000 in Odyssey blocking buffer for 30 min then wash three times in 1×TBS-Tween. Signals will be quantified using the Odyssey Infrared Imaging System (LI-COR Bioscience). The resulting densitometric readings will be used to generate a ratio of phopho-protein to α-tubulin or GAPDH. The resulting ratio will be further normalized to vehicle treated control group.

Compound Analysis: For determination of compound levels in the tissue preparations, 70% isopropyl alcohol at a ratio of 3:1 v:w (μL:mg) will be added to the tissue samples followed by homogenization with a bead beater (Biospec). Proteins will be precipitated from 50 μL of the resulting tissue homogenate in 150 μL of acetonitrile containing of the internal standard (tolbutamide) followed by centrifugation at 3000 rpm for 10 min. One hundred microliters of the resulting supernatant will be added to 100 μL of water, mixed well and injected on the LC-MS/MS system using the following program for assessing compound levels:
Phenomenex LUX Cellulose column (4.6×150 mm, 5 μm)
Mobile Phase A—0.1% Formic Acid in Water
Mobile Phase B—0.1% Formic Acid in Acetonitrile
Gradient:
   Initial—40% A
   2 minutes—40% A
   2.1 minutes—2% A
   3 minutes—2% A
   3.1 minutes—40% A
   4 minutes—40% A
Flow rate 0.8 mL/min
Column Temp 40 degrees C.
Sciex 5500 Triple Quad Mass Spec

### Example E: The Effect of a Single Dose of Compound I as a Lithium Chloride Salt or Co-Crystal on the Sucrose Preference and Novelty Suppressed Feeding Tests and Synaptic Protein Expression

Study Design: Male rats weighing between 175 and 200 g will be randomized into four (4) study groups, following a 5-day acclimation period. On study day minus 20, two (2) groups of rats will be subjected to CUS for 25 days and two (2) groups of rats will be housed normally serving as the NS group. On the day 21 of the CUS protocol, rats will receive a single dose of either Veh or compound I as a lithium chloride co-crystal (160 mg/kg) by oral gavage (day 0). The SPT and NSFT will be performed 24 and 48 hrs post-administration (days 1 and 2), respectively. Upon completion of the behavioral tests, after 25 days of the CUS protocol a second dose of compound I as a lithium chloride salt or co-crystal or Veh will be administered on day 5 and rats will be sacrificed 24 hrs later by conscious decapitation. Crude synaptosomes will be prepared from the PFC and the synaptic proteins, GluR1 and PSD95 will be quantitated by WB.

Formulation of Compound I as a Lithium Chloride Salt or Co-Crystal (50 mg/mL): Compound I as a lithium chloride salt or co-crystal will be prepared by dissolving in Veh to a concentration of 50 mg/mL. The solution will be administered to rats in Groups 2 and 4 by oral gavage at a volume of 10 mL/kg for a final dose of 160 mg/kg. An equivalent volume of Veh will be administered to Groups 1 and 3.

Prefrontal Cortex Synaptosomes Preparations: The brain will be dissected from rats and rinsed in PBS. The PFC will be collected and homogenized at 4° C. in homogenization buffer (0.32 M sucrose, 20 mM HEPES at pH 7.4, 1 mM EDTA, 5 mM NaF, 1 mM NaVO₃ and protease inhibitor cocktail (Roche; Ser. No. 19/543,200)). The homogenate will be centrifuged for 10 min at 2,800 rpm at 4° C. after which the supernatant will be removed and re-centrifuged at 12,000 rpm for 10 min at 4° C. The resulting pellet will be re-suspended in Lysis buffer (50 mM Tris-HCl (pH 7.5), 150 mM NaCl, 1% Triton X-100, 0.1% SDS, 2 mM, EDTA. 1 mM NaVO₃, 5 mM NaF and protease inhibitor cocktail) and sonicated on ice for 20 s at 50% amplitude. The protein concentration will be determined by Bradford assay and all samples will be mixed with loading buffer (60 mM Tris-HCl pH 6.8, 20 mM DTT, 2% SDS, 10% glycerol, 5% β-mercaptoethanol and 0.01% Bromophenol blue) and stored at −20° C. until WB analysis.

Western Blot Analysis: Western blot analysis for GluR1, PSD95 and GAPDH will be performed as previously described (Li, N. et al., Science 329(5994): 959-964 (2010)). Briefly, synaptosomes (15 μg protein) will be loaded into 10-15% SDS PAGE gel for electrophoresis and transferred using transfer to a polyvinylidene difluoride (PVDF) membrane in transfer buffer (10× premixed electrophoresis buffer containing 25 mM Tris, 192 mM glycine, pH 8.3: Bio-Rad). The PVDF membranes will be blocked for 1 hr at room temperature with blocking buffer (2% BSA in PBS-T (10 mM Phosphate, pH 7.4, 2.7 mM KCl, 137 mM NaCl and 0.1% Tween-20)) and subsequently incubated with primary antibodies: rabbit anti-GluR1 at 1:1000 (Cell Signaling: #13185), rabbit anti-PSD95 at 1:1000 (Cell Signaling; #9644) and rabbit anti-GAPDH at 1:1000 (cell signaling: #5174) in blocking buffer overnight at 4° C. The next day, membranes will be washed 3 times in PBS-T buffer, and incubated with horseradish peroxidase conjugated anti-mouse or anti-rabbit secondary antibody at 1:5000 to 1:10000 (Vector Laboratories Inc) for 1 hr. After final three washes with PBS-T buffer, bands will be detected using enhanced chemiluminescence. The blots then will be incubated in the stripping buffer (2% SDS, 100 mM β-mercaptoethanol, 50 mM Tris pH 6.8) for 30 min at 50-55° C. followed by three washes with PBS-T buffer. The stripped blots will be kept in blocking solution for 1 hr and incubated with the primary antibody directed against GAPDH for loading control. Densitometric analysis of total immunoreactivity for each protein will be conducted using NIH Image J software. The resulting densitometric readings will be used to generate a ratio of total protein to GAPDH. The resulting ratio will be further normalized to NS-Veh group for each protein.

### Example F: The Dependence on mTORC1 Activation for the Pharmacological Activity of Compound I as a Lithium Chloride Salt or Co-Crystal in the Forces Swimming and the Novelty Suppressed Feeding Tests Following a Single Oral Administration in Rats

Study Design: Male rats weighing between 175 and 200 g will be randomized into three (3) study groups, following a 5-day acclimation period. All rats will be surgically implanted with bilateral IT cannulas in the PFC 2 weeks prior to dosing. On the day of dosing, all treatment groups will receive bilateral IT infusions (0.5 μL/side) containing either the rapamycin (R) vehicle (Veh-R, 10% DMSO) or rapamycin (R, 0.01 nmol/L) which has been previously shown to completely inhibit mTORC1 activity. Thirty minutes following the intrathecal infusions, either compound I vehicle (Veh-NV, 0.5% methylcellulose/0.1% Tween-80) or compound I as a lithium chloride salt or co-crystal (160 mg/kg) will be administered by oral gavage. Each treatment group will be assessed at the designated time following oral dosing in FST 24 hrs (day 1) LMA 48 hrs (day 2) and the NSFT 72 hrs (day 3 following a 20 hr period of fasting). LMA will be measured to rule out overall changes in generally locomotor activity.

Preparation of Test Articles: Rapamycin (Cell Signaling; #9904) will be prepared in a solution of 10% DMSO (Veh-R) to a final concentration of 10 M. R or Veh-R will be administered bilaterally (0.005 nmol/0.5 μL per side) into the medial PFC via IT infusion 30 min prior to treatment with compound I as a lithium chloride salt or co-crystal or Veh-NV by oral gavage. Compound I as a lithium chloride salt or co-crystal will be prepared by dissolving in Veh (0.5% methylcellulose/0.1% Tween-80) at a concentration of 50 mg/mL. The dosing volume based on the weight of the animal (3.2 mL/kg) will be administered by oral gavage to study animals in Groups 2 and 3.

Surgical Procedure and Administration of Rapamycin: Rats will be stereotactically implanted with a guide cannula (22GA) into the medial PFC (coordinates from bregma: +3.2 AP, ±1.0 ML, −3.5 DV from dura). Surgical procedures will be carried out under the anesthesia Nembutal (i.p. 55 mg/kg). Postoperative care will consist of in peri-surgical administration of carprofen (5 mg/kg) and topical triple antibiotic. After a 2-week recovery period, R (0.01 nmol in 1 μL for PFC infusion) or Veh-R will be delivered at the rate of 0.25 μL/min with an injection cannula (26GA) protruding 0.5 mm beyond the guide cannula 30 min before oral administration of compound I as a lithium chloride salt or co-crystal or Veh-NV. The dose of rapamycin will be chosen based on previous reports demonstrating effective and selective inhibition of mTORC1 activity.

### Example G: Duration of Behavioral Changes in the Forces Swimming Test and Novelty Suppressed Feeding Test after a Single Dose of Compound I as a Lithium Chloride Salt or Co-Crystal or Ketamine

Study Design: Male rats weighing between 175 and 200 g will be randomized into six (6) study groups, following a 5-day acclimation period. A single dose of all test articles will be administered on day 0 and the behavioral tests will be performed 3, 7 and 10 days later. Groups 1 and 2 will be dosed on day 0 with compound I as a lithium chloride salt or co-crystal (160 mg/kg by oral gavage) and Ket (10 mg/kg by i.p. injection), respectively and subjected to a FST on day 3. The rats in Group 3 and 4 will receive a single dose of the compound I vehicle (Veh) or compound I as a lithium chloride salt or co-crystal (160 mg/kg), respectively, by oral gavage on day 0. The rats in Group 5 and 6 will receive a single dose of the Ket vehicle (Sal) or Ket (10 mg/kg), respectively, by i.p. injection on day 0. The rats in groups 3-6 will be subjected to a FST on day 7 and a NSFT on day 10. All rats in groups 3-6 will be fasted the night before the NSFT for 20 hours.

### Example H: Physiological Changes in the Layer V Pyramidal Neurons after a Single Dose of Compound I as a Lithium Chloride Salt or Co-Crystal

Study Design: Male rats weighing between 175 and 200 g will be randomized into two (2) study groups, following a 5-day acclimation period. On study day 0, rats will receive a single dose of either NV vehicle (Veh, 0.5% methylcellulose/0.11% Tween-80) or compound I as a lithium chloride salt or co-crystal (160 mg/kg), by oral gavage. On day 1, 24 hrs after dosing, rats will be sacrificed, and brain slices will be prepared and subjected to whole cell patch clamp recording of layer V pyramidal neurons in PFC.

Preparation of Test Articles: Compound I as a lithium chloride salt or co-crystal will be prepared by dissolving in Veh (0.5% methylcellulose/0.1% Tween-80) at a concentration of 50 mg/mL. The dosing volume based on the weight of the animal (3.2 mL/kg) will be administered by oral gavage to study animals. Test articles will be prepared the day of dosing.

Brain Slice Preparation: Brain slices will be prepared according to published procedures (Liu, R. J. et al., J. Neurosci. 22(21): 9453-9464 (2002)). Briefly, rats will be anesthetized with chloral hydrate (400 mg/kg, i.p.), in adherence to protocols approved by the Yale Animal Care and Use Committee. After decapitation, the brains will be removed rapidly and placed in ice-cold (4° C.) artificial cerebrospinal fluid (ACSF) in which sucrose (252 mM) will be substituted for NaCl (sucrose-ACSF) to prevent cell swelling. A block of tissue containing PFC will be dissected and coronal slices (400 μm) will be cut in sucrose-ACSF with an oscillating-blade tissue slicer (Leica VT1000S). After placement of the slice in a submerged recording chamber, the bath temperature will be raised to 32° C. Known concentrations of drugs dissolved in ACSF, applied through a stopcock arrangement at a fast flow rate (˜4 mL/min), will reach the slice within 7-10 s. The standard ACSF (pH=7.35) will be equilibrated with 95% O₂/5% CO₂ and contain 128 mM NaCl. 3 mM KCl, 2 mM CaCl₂), 2 mM MgSO₄, 24 mM NaHCO₃, 1.25 mM NaH₂PO₄, and 10 mM, D-glucose. A recovery period of ˜1-2 h will be allowed before commencement of recording.

Electrophysiology Recording: Pyramidal neurons in layer V will be visualized by video microscopy using an Olympus BX50WI microscope (×60 IR lens) with infrared differential interference contrast (IR/DIC) video microscopy (Olympus), according to published procedures (Lambe, E. K. & Aghajanian, G. K. Neuron 40(1):139-150 (2003)). Low-resistance patch pipettes (3-5 MΩ) will be pulled from patch-clamp glass tubing (Warner Instruments) by using a Flaming-Brown Horizontal Puller (model P-97: Sutter Instruments). The pipettes will be filled with the following solution: 115 mM K gluconate, 5 mM KCl, 2 mM MgCl₂, 2 mM Mg-ATP, 2 mM Na₂ATP, 10 mM Na₂-phosphocreatine, 0.4 mM Na₂GTP, and 10 mM HEPES, pH 7.33. Neurobiotin (0.3%) will be added to the pipette solution to mark cells for later imaging. Whole-cell recordings will be performed with an Axoclamp-2B amplifier (Axon Instruments). The output signal will be low-pass-filtered at 3 KHz, amplified×100 through Cyberamp, digitized at 15 kHz, and will be acquired by using pClamp 9.2/Digidata 1320 software (Axon Instruments). Series resistance, monitored throughout the experiment, will be usually between 4 and 8 MΩ. To minimize series resistance errors, cells will be discarded if series resistance rose above 10Ω. Postsynaptic currents will be studied in the continuous single-electrode voltage-clamp mode (3000 Hz low-pass filter) clamped near resting potential (75 mV±5 mV) to minimize holding currents. After completion of recording, slices will be transferred to 4% paraformaldehyde in 0.1 M phosphate buffer and stored overnight at 4° C. Slices then will be processed with streptavidin conjugated to Alexa 594 (1:1000; Invitrogen) for Neurobiotin visualization in labeled cells.

Spine Density Analysis: Labeled neurons within layer V of anterior cingulate (Cgl) and prelimbic mPFC (Cg3) will be imaged with a two-photon Ti:sapphire laser scanning system for analysis of spine density and morphology (810 nanometers: Mai Tai, Spectra Physics, Mountain View, California) coupled to direct detection Radiance 2000 BioRad laser scanner (Zeiss Micromaging, Thomwood, New York) mounted on a Olympus BX50WI microscope, using a 60× (0.9 numerical aperture) water-immersion objective. This will include the total number of spines on proximal and distal tufts of layer V neurons, as well as the spine head diameter, and indication of spine maturation. The length of apical tuft branch segments will be determined within the 3D matrix of each Z-stack by using Neurolucida 10.2 (MicroBrightField). Spine density and spine head diameter analysis are done with the Autospine module of Neurolucida Explorer (version 10.2) on the raw image stacks (2-5 optical sections, 1 μm apart). Spine density and segmentation (beading) will be sampled in three zones: tips of tuft branches as they approach the pial membrane, intermediate dendrites approximately midway between the pial membrane and bifurcation of apical trunk, and proximal tuft dendrites just distal to the bifurcation. Results will be expressed in terms of total dendritic length, spine density, and segmentation density. Results will be expressed in terms of spine density per 10 μm.

### Example I: Behavioral Changes in the Forced Swim and Novelty Suppressed Feeding Tests after Daily Dosing of Compound I as a Lithium Chloride Salt or Co-Crystal or Every-Other-Day Dosing of Ketamine

Study Design: Male Sprague Dawley rats weighing between 175 and 200 g will be randomized into four (4) study groups, following a 5-day acclimation period. On study day minus 1 rats will be subjected to a pre-swim. Beginning on study day 0, rats in Group 2 will receive a dose of Ket (10 mg/kg) on alternate days (day 0, 2, 4, and 6) each by i.p. injection. The rats in Groups 1 will receive a daily dose of Veh by oral gavage for 7 days (days 0 to 6). The rats in Groups 3 and 4 will receive a daily dose of compound I as a lithium chloride salt or co-crystal (40 or 80 mg/kg) for 7 days (days 0 to 6) each by oral gavage. All rats will be subjected to the FST on day 7, 24 hrs after last dosing. On day 8, 48 hrs after last dosing, the LMA of all rats will be measured in an open field. Rats then will be fasted for 20 hrs and subjected to the NSFT on day 9 (72 hrs post-last dose).

Preparation of Test Articles: Ket (Sigma, cat #K1884) will be dissolved in Sal at a concentration of 10 mg/mL. Injection volumes (i.p) of 1 mL/kg of Ket will be administered to Group 2. Compound I as a lithium chloride salt or co-crystal will be prepared by dissolving in Veh (0.5% methylcellulose/0.1% Tween-80) at a concentration of 50 mg/mL. The dosing volume based on the weight of the animal (3.2 mL/kg) of Veh or compound I as a lithium chloride salt or co-crystal will be administered daily by oral gavage to study animals in Groups 1, 3, and 4, respectively. Test articles will be prepared the day of dosing.

### Example J: Marmoset Human Threat Test

Study Design: Marmosets (Callithrix jacchus) will be paired and divided randomly divided into treatment groups. Twenty-four hours before the Human Threat Test (HTT) animals will be treated with vehicle, ketamine (0.3 mg/kg; i.m.) or compound I as a lithium chloride salt or co-crystal (160 mg/kg; p.o.). The following day, the same animals will be treated with either vehicle (s.c.) or chloriazepoxide (1 mg/kg; s.c.). The animals then will be then monitored for the number of threat postures over a two (2) minutes period with the presence of a human observer. Locomotor activity will be monitored over the same period, as measured by the number of jumps observed.

While we have described a number of embodiments of this invention, it is apparent that our basic examples may be altered to provide other embodiments that utilize the compound and methods of this invention. Therefore, it will be appreciated that the scope of this invention is to be defined by the appended claims rather than by the specific embodiments that have been represented by way of example.

We claim:

1. A compound having the formula LiCl*compound I, or a solvate or hydrate thereof, wherein compound I has the structure:

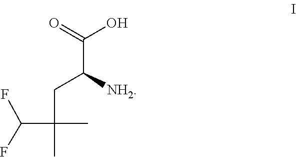

2. The compound of claim 1, wherein the LiCl*compound I is a co-crystal.

3. The compound of claim 1, wherein the lithium and the compound I are in a stoichiometric ratio of about 1 to about 1.

4. A pharmaceutical composition comprising the compound according to claim 1 and a pharmaceutically acceptable carrier, adjuvant, or vehicle.

5. The pharmaceutical composition according to claim 4, wherein the composition is formulated for oral administration.

6. The pharmaceutical composition according to claim 4, further comprising an additional therapeutic agent.

7. A method of treating, preventing, and/or reducing a risk of a disease, disorder, or condition mediated by mTORC1 in a patient comprising administering to a patient in need thereof an effective amount of the compound according to claim 1 or the pharmaceutical composition according to claim 4.

8. The method of claim 7, wherein the disease, disorder, or condition mediated by mTORC1 is depression, bipolar disorder, schizophrenia, chronic unpredictable stress, autism, lysosomal storage disease, Batten disease, cystinosis, Fabry disease, mucolipidosis, mental retardation, anorexia, bulimia, anemia, neutropenia, headache, alcoholism, post-traumatic stress disorder (PTSD), epilepsy, diabetes, liver disease, kidney disorders, arthritis, skin conditions such as seborrhea, overactive thyroid, asthma, Huntington's disease, Graves' disease, herpes simplex, movement disorders such as tardive dyskinesia, Tourette's syndrome, cyclical vomiting, Meniere's disease, tingling or crawling sensation in the skin (paresthesias), or aggressive behavior in attention deficit-hyperactivity disorder (ADHD).

9. A method of treating treatment-resistant depression in a patient in need thereof, said method comprising administering to said patient an effective amount of the compound according to claim 1 or the pharmaceutical composition according to claim 4.

10. A method of treating major depressive disorder in a patient in need thereof, said method comprising administering to said patient an effective amount of the compound according to claim 1 or the pharmaceutical composition according to claim 4.
